(19) United States
(12) Patent Application Publication
(10) Pub. No.: US 2018/0306810 A1
(43) Pub. Date: Oct. 25, 2018

(54) ANTIBODIES RECOGNIZING PHOSPHORYLATION AT SPECIFIC AMINO ACID RESIDUES OF TRANSACTIVE RESPONSE DNA-BINDING PROTEIN 43

(71) Applicant: Saint Louis University, St. Louis, MO (US)

(72) Inventor: Yuna Mary Ayala, Clayton, MO (US)

(73) Assignee: Saint Louis University, St. Louismo, MO (US)

(21) Appl. No.: 15/738,015

(22) PCT Filed: Jun. 16, 2016
(86) PCT No.: PCT/US2016/037881

§ 371 Date: Dec. 19, 2017

Related U.S. Application Data
(60) Provisional application No. 62/181,840, filed on Jun. 19, 2015.

(51) Int. Cl. G01N 33/68; G01N 33/53; C07K 16/28

(52) U.S. Cl. CPC G01N 33/6896; G01N 33/53; G01N 2800/28; G01N 2440/14; C07K 16/28

(57) ABSTRACT

Disclosed are antibodies and methods of using the antibodies to detect Transactive response DNA-binding protein 43. In particular, the present disclosure discloses antibodies that specifically bind to phosphorylated epitopes of TDP-43 and methods for using the antibodies to detect phosphorylated epitopes of TDP-43.

### CROSS-REFERENCE TO RELATED APPLICATIONS

This application claims priority to U.S. Provisional Application No. 62/181,840, filed on Jun. 19, 2015, the disclosure of which is incorporated by reference in its entirety.

### STATEMENT OF GOVERNMENT SUPPORT

This invention was made with government support under NS082391 awarded by the National Institutes of Health. The government has certain rights in the invention.

### INCORPORATION OF SEQUENCE LISTING

A paper copy of the Sequence Listing and a computer readable form of the Sequence Listing containing the file named “SLU 15-024_ST25.txt”, which is 4,164 bytes in size (as measured in MICROSOFT WINDOWS® EXPLORER), are provided herein and are herein incorporated by reference. This Sequence Listing consists of SEQ ID NO:1-2.

### BACKGROUND OF THE DISCLOSURE

The present disclosure relates generally to antibodies and methods of using the antibodies to detect Transactive response DNA-binding protein 43. In particular, the present disclosure discloses antibodies that specifically bind to phosphorylated epitopes of TDP-43 and methods for using the antibodies to detect phosphorylated epitopes of TDP-43.

Transactive response DNA-binding protein 43, more commonly known as TDP-43, is a protein strongly connected to various types of neurodegenerative diseases. Mutations in the TDP-43 gene are dominantly linked to amyotrophic lateral sclerosis (ALS) or Lou Gehrig's disease. The characteristic features of TDP-43 pathology in the associated neurodegenerative disorders are protein aggregation into insoluble bodies and protein modifications that include ubiquitination, phosphorylation, and cleavage of TDP-43 into smaller fragments. These occur in the brain, spinal cord and muscle of patients and present in approximately 98% of all ALS, 50% of frontotemporal lobar degeneration, Alzheimer's disease, chronic traumatic encephalopathy (also known as traumatic brain injury), neuronal injury, and more rare disorders, such as inclusion body myopathy associated with Paget's disease of the bone and fronto-temporal dementia (IBMPFD), and limb-girdle muscular dystrophy (LGMD). Various ideas have been put forward to explain the molecular link between TDP-43, neuronal cell death and disease. Protein aggregation may be toxic to the affected cells, although neurotoxicity may be achieved in the absence of detectable TDP-43 aggregation in model systems. Aggregation is accompanied by sequestration of the protein from the rest of the cell suggesting that this is a loss of function mechanism. Finally, mounting evidence shows that relatively modest changes in TDP-43 levels affects expression of a great number of genes in a dose dependent manner and promotes neurotoxicity.

TDP-43 is a predominantly nuclear RNA-binding protein that controls RNA processing and gene expression of hundreds of genes. TDP-43 modulates gene expression mostly through directly binding RNA transcripts. The critical role of this protein is highlighted in mice, where it is lethal upon deletion, and in all other model organisms tested to date, which exhibit large functional impairments in the absence of TDP-43. Despite this essential role of TDP-43 very little is known about how its function is regulated and the cellular mechanisms involved. Accordingly, there exists a need to understand the molecular mechanisms of TDP-43 as a target for therapeutic treatment and develop reagents for detecting TDP-43.

### BRIEF DESCRIPTION OF THE DISCLOSURE

The present disclosure relates generally to antibodies and methods of using the antibodies to detect Transactive response DNA-binding protein 43. In particular, the present disclosure discloses antibodies that specifically bind to phosphorylated epitopes of TDP-43 and methods for using the antibodies to detect phosphorylated epitopes of TDP-43.

In one aspect, the present disclosure is directed to isolated antibodies that specifically bind to a phosphorylated epitope of Transactive response DNA-binding protein 43 (TDP-43).

In another aspect, the present disclosure is directed to a method for detecting TDP-43 in a sample, the method comprising: contacting a sample with an antibody that specifically binds to a phosphorylated epitope of TDP-43, wherein the phosphorylated epitope of TDP-43 is selected from the group consisting of Thr88, Ser91, Ser92, Ser125, Thr126, Thr153, Tyr155, Thr157, Ser180, Ser183, Ser292, and combinations thereof, wherein the antibody and TDP-43 forms a complex; and detecting the complex.

In another aspect, the present disclosure is directed to methods for diagnosing a TDP-43-associated neurodegenerative disorder in a subject having or suspecting of having the TDP-43-associated neurodegenerative disorder, the method comprising: obtaining a sample from the subject; determining phosphorylation of TDP-43 in a sample by contacting the sample with an antibody that specifically binds a phosphorylated epitope of TDP-43, wherein the phosphorylated epitope of TDP-43 is selected from the group consisting of Thr88, Ser91, Ser92, Ser125, Thr126, Thr153, Tyr155, Thr157, Ser180, Ser183, Ser292, and combinations thereof; wherein the antibody and TDP-43 forms a complex; and detecting the complex.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a schematic illustration depicting phosphorylated TDP-43 amino acid residues identified by mass spectrometry. The nuclear localization signal (NLS) is at the N-terminus and the bipartite consensus sequence flanks Thr88, Ser91, Ser92. The RNA recognition motif 1 (RRM1) contains five phosphorylation sites and two residues were identified in the linker region between the two RRMs.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In one aspect, the present disclosure is directed to an isolated antibody that specifically binds to a phosphorylated epitope of Transactive response DNA-binding protein 43 (TDP-43). The phosphorylated epitope of TDP-43 is selected from Thr88, Ser91, Ser92, Ser125, Thr126, Thr153, Tyr155, Thr157, Ser180, Ser183, Ser292, and combinations thereof.

Particularly suitable antibodies can be an antibody that specifically binds phosphorylated Thr88 of TDP-43; an antibody that specifically binds phosphorylated Ser91 of TDP-43; an antibody that specifically binds phosphorylated Ser92 of TDP-43; an antibody that specifically binds the combination of phosphorylated Thr88, phosphorylated Ser91 and phosphorylated Ser92 of TDP-43; an antibody that specifically binds phosphorylated Ser125 and phosphorylated Thr126 of TDP-43; an antibody that specifically binds phosphorylated Thr153 and phosphorylated Tyr155 of TDP-43; an antibody that specifically binds phosphorylated Thr153 of TDP-43; an antibody that specifically binds phosphorylated Tyr155 of TDP-43; an antibody that specifically binds phosphorylated Thr157 of TDP-43; an antibody that specifically binds phosphorylated Thr153 and phosphorylated Tyr155 of TDP-43; an antibody that specifically binds phosphorylated Tyr155 and phosphorylated Thr157 of TDP-43; an antibody that specifically binds phosphorylated Ser180 and phosphorylated Ser183 of TDP-43; an antibody that specifically binds phosphorylated Ser180 of TDP-43; an antibody that specifically binds phosphorylated Ser183 of TDP-43; and an antibody that specifically binds phosphorylated Ser292 of TDP-43. Epitope numbering of TDP-43 is in reference to accession number NP_031401.1 (see, SEQ ID NO:1).

As used herein, “antibody” and “antibodies” refer to polyclonal antibodies, monoclonal antibodies, multispecific antibodies, human antibodies, humanized antibodies, camelised antibodies, chimeric antibodies, single-chain Fvs (scFv), single chain antibodies, single domain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. Antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “chimeric antibody” refers to a polypeptide comprising at least the antigen binding portion of an antibody molecule linked to at least part of another protein (typically an immunoglobulin constant domain).

As used herein, “humanized antibody” refers to an immunoglobulin variant or fragment thereof, that is capable of binding to a predetermined antigen and which includes framework regions having substantially a human immunoglobulin amino acid sequence and Complementarity Determining Regions (CDRs) having substantially a non-human immunoglobulin amino acid sequence. A humanized antibody has one or more amino acid residues introduced into it from a source that is non-human. In general, the humanized antibody will include substantially all of at least one, and typically two, variable domains (Fab, Fab′, F(ab′)2, Fabc, Fv) in which all or substantially all of the CDR regions correspond to those of a non-human immunoglobulin and all or substantially all of the framework regions are those of a human immunoglobulin consensus sequence. The humanized antibody can include at least a portion of an immunoglobulin constant region (Fc), typically that of a human immunoglobulin. Generally, the antibody contains both the light chain and at least the variable domain of a heavy chain. The humanized antibody can be selected from any class of immunoglobulins, including IgM, IgG, IgD, IgA and IgE, and any isotype, including IgG1, IgG2, IgG3 and IgG4. The humanized antibody can include sequences from more than one class or isotype, and selecting particular constant domains to include desired effector functions is within the ordinary skill in the art.

As used herein, “specifically binds to a phosphorylated epitope of TDP-43” refers to peptides, polypeptides, proteins, fusion proteins, and antibodies that specifically bind to at least one phosphorylated amino acid of TDP-43 and do not specifically bind to other peptides or the amino acid of TDP-43 in its unphosphorylated form. Antibodies and antibody fragments that specifically bind to a phosphorylated epitope of TDP-43 can be identified, for example, by immunoassays, BIAcore kinetic assay, and other techniques known to those of skill in the art. An antibody binds specifically to a phosphorylated epitope of TDP-43 when it binds to a phosphorylated epitope of TDP-43 with higher affinity than to any cross-reactive antigen as determined using experimental techniques, such as radioimmunoassays (RIA), Western blot analysis, and enzyme-linked immunosorbent assay (ELISA), immunoprecipitation, immunohistochemistry, and combinations thereof.

As used herein, “epitope” refers to sites or fragments of a polypeptide or protein having antigenic or immunogenic activity in an animal including mammals, and particularly in humans. As such, “phosphorylated epitope” refers to sites or fragments of a polypeptide or protein having antigenic or immunogenic activity in an animal, in one aspect, in a mammal, and, in one aspect, in a human, and which contains at least one phosphorylated amino acid. An epitope having immunogenic activity is a site or fragment of a polypeptide or protein that elicits an antibody response in an animal. An epitope having antigenic activity is a site or fragment of a polypeptide or protein to which an antibody immunospecifically binds as determined by any method well-known to one of skill in the art, for example by immunoassays such as radioimmunoassays (RIA), Western blot analysis, and enzyme-linked immunosorbent assay (ELISA), immunoprecipitation, immunohistochemistry, and combinations thereof.

The antibody can further be conjugated to a detectable agent. Suitable detectable agents include fluorescent agents, biotin, streptavidin, avidin, Neutravidin, gold beads, and enzymes. Detectable agents can be covalently attached to the antibody using methods known to those skilled in the art including using commercially available kits.

To generate antibodies to TDP-43, a full length TDP-43 polypeptide or fragment thereof can be prepared using well known recombinant DNA technology methods such as those set forth in Sambrook et al. (Molecular Cloning: A Laboratory Manual, Fourth Edition, Cold Spring Harbor Laboratory Press, Cold Spring Harbor, N.Y. 2012) and/or Ausubel et al., eds, (Current Protocols in Molecular Biology, Green Publishers Inc. and Wiley and Sons, NY 1994). A gene or cDNA encoding a TDP-43 protein or fragment thereof may be obtained for example by screening a genomic or cDNA library, or by PCR amplification. Probes or primers useful for screening the library can be generated based on sequence information for other known genes or gene fragments from the same or a related family of genes. In addition, where a TDP-43 gene has been identified from one species, all or a portion of that gene may be used as a probe to identify homologous genes from other species. The probes or primers may be used to screen cDNA libraries from various tissue sources believed to express the TDP-43 gene. Typically, conditions of high stringency are employed for screening to minimize the number of false positives obtained from the screen.

Chemical synthesis can also be used to prepare a gene encoding a TDP-43 polypeptide or fragment thereof using methods well known to those skilled in the art such as those described by Engels et al. (Angew. Chem. Intl. Ed., 1989, 28:716-734). These methods include, for example, the phosphotriester, phosphoramidite, and H-phosphonate methods for nucleic acid synthesis. A preferred method for such chemical synthesis is polymer-supported synthesis using standard phosphoramidite chemistry. Typically, the DNA encoding the TDP-43 polypeptide can be several hundred nucleotides in length. Nucleic acids larger than about 100 nucleotides can be synthesized as several fragments using these methods. The fragments can then be ligated together to form the full length TDP-43 polypeptide.

Nucleic acid variants (wherein one or more nucleotides are designed to differ from the wild-type or naturally occurring TDP-43) can be produced using site directed mutagenesis, PCR amplification, chemical synthesis and other appropriate methods, where the primer(s) have the desired point mutations. Preferred nucleic acid variants include those containing nucleotide substitutions accounting for codon preference in the host cell that is to be used to produce TDP-43.

The TDP-43 gene, cDNA, or fragment thereof can be inserted into an appropriate expression or amplification vector using standard ligation techniques. The vector is typically selected to be functional in the particular host cell employed (i.e., the vector is compatible with the host cell machinery such that amplification of the TDP-43 gene and/or expression of the gene can occur). The TDP-43 gene, cDNA or fragment thereof may be amplified/expressed in prokaryotic, yeast, insect and eukaryotic host cells. For generating phosphorylated TDP-43, yeast, insect, and mammalian host cells are preferable.

The TDP-43 polypeptides, fragments, variants, and/or derivatives can be used to prepare antibodies using standard methods. Thus, antibodies that react with the phosphorylated epitopes of the TDP-43 polypeptides, as well as reactive fragments of such antibodies, are also contemplated as within the scope of the present disclosure. Suitable antibodies include polyclonal, monoclonal, recombinant, chimeric, single-chain and bispecific. Suitable antibodies and fragments thereof can either be of human origin, or can be “humanized” (i.e., prepared so as to prevent or minimize an immune reaction to the antibody when administered to a patient). The antibody fragment may be any fragment that is reactive with the TDP-43 polypeptides of the present disclosure, such as, Fab, Fab′, etc.

In another aspect, the present disclosure is directed to mixtures of antibodies that specifically bind a phosphorylated epitope of TDP-43, as disclosed herein.

In another aspect, the present disclosure is directed to a method for detecting at least one phosphorylated epitope of TDP-43 in a sample. The method includes: contacting a sample with an antibody that specifically binds to a phosphorylated epitope of TDP-43, wherein the phosphorylated epitope of TDP-43 is selected from the group consisting of Thr88, Ser91, Ser92, Ser125, Thr126, Thr153, Tyr155, Thr157, Ser180, Ser183, Ser292, and combinations thereof, wherein the antibody and TDP-43 forms a complex; and detecting the complex.

Particularly suitable antibodies can be an antibody that specifically binds phosphorylated Thr88 of TDP-43; an antibody that specifically binds phosphorylated Ser91 of TDP-43; an antibody that specifically binds phosphorylated Ser92 of TDP-43; an antibody that specifically binds the combination of phosphorylated Thr88, phosphorylated Ser91 and phosphorylated Ser92 of TDP-43; an antibody that specifically binds phosphorylated Ser125 and phosphorylated Thr126 of TDP-43; an antibody that specifically binds phosphorylated Thr153 and phosphorylated Tyr155 of TDP-43; an antibody that specifically binds phosphorylated Thr153 of TDP-43; an antibody that specifically binds phosphorylated Tyr155 of TDP-43; an antibody that specifically binds phosphorylated Thr157 of TDP-43; an antibody that specifically binds phosphorylated Thr153 and phosphorylated Tyr155 of TDP-43; an antibody that specifically binds phosphorylated Tyr155 and phosphorylated Thr157 of TDP-43; an antibody that specifically binds phosphorylated Ser180 and phosphorylated Ser183 of TDP-43; an antibody that specifically binds phosphorylated Ser180 of TDP-43; an antibody that specifically binds phosphorylated Ser183 of TDP-43; and an antibody that specifically binds phosphorylated Ser292 of TDP-43. Epitope numbering of TDP-43 is in reference to accession number NP_031401.1 (see, SEQ ID NO:1).

In another aspect, the present disclosure is directed to methods for diagnosing a TDP-43-associated neurodegenerative disorder. The method includes obtaining a sample from the subject; determining phosphorylation of TDP-43 in a sample by contacting the sample with an antibody that specifically binds a phosphorylated epitope of TDP-43, wherein the phosphorylated epitope of TDP-43 is selected from the group consisting of Thr88, Ser91, Ser92, Ser125, Thr126, Thr153, Tyr155, Thr157, Ser180, Ser183, Ser292, and combinations thereof; wherein the antibody and TDP-43 forms a complex; and detecting the complex.

Particularly suitable antibodies can be an antibody that specifically binds phosphorylated Thr88 of TDP-43; an antibody that specifically binds phosphorylated Ser91 of TDP-43; an antibody that specifically binds phosphorylated Ser92 of TDP-43; an antibody that specifically binds the combination of phosphorylated Thr88, phosphorylated Ser91 and phosphorylated Ser92 of TDP-43; an antibody that specifically binds phosphorylated Ser125 and phosphorylated Thr126 of TDP-43; an antibody that specifically binds phosphorylated Thr153 and phosphorylated Tyr155 of TDP-43; an antibody that specifically binds phosphorylated Thr153 of TDP-43; an antibody that specifically binds phosphorylated Tyr155 of TDP-43; an antibody that specifically binds phosphorylated Thr157 of TDP-43; an antibody that specifically binds phosphorylated Thr153 and phosphorylated Tyr155 of TDP-43; an antibody that specifically binds phosphorylated Tyr155 and phosphorylated Thr157 of TDP-43; an antibody that specifically binds phosphorylated Ser180 and phosphorylated Ser183 of TDP-43; an antibody that specifically binds phosphorylated Ser180 of TDP-43; an antibody that specifically binds phosphorylated Ser183 of TDP-43; and an antibody that specifically binds phosphorylated Ser292 of TDP-43. Epitope numbering of TDP-43 is in reference to accession number NP_031401.1 (see, SEQ ID NO:1).

The terms “subject” and “patient” are used interchangeably herein to refer to an animal, including, mammals such as cows, pigs, horse, cats, dogs, rats, mice, primates, and humans.

In one aspect, the subject has a neurodegenerative disorder. In another aspect, the subject is suspected of having a neurodegenerative disorder.

TDP-43-associated neurodegenerative disorders can be, for example, amyotrophic lateral sclerosis, frontotemporal lobar degeneration, Alzheimer's disease, inclusion body myositis, Inclusion body myopathy associated with Paget's disease of the bone and frontotemporal dementia (IBMPFD, OMIM 167320), limb-girdle muscular dystrophy (LGMD), neuronal injury, and chronic traumatic encephalopathy.

The phosphorylation of TDP-43 is determined by detecting a phosphorylated epitope of TDP-43. The phosphorylated epitope of TDP-43 is selected from Thr88, Ser91, Ser92, Ser125, Thr126, Thr153, Tyr155, Thr157, Ser180, Ser183, Ser292, and combinations thereof, as described herein.

Particularly suitable antibodies can be an antibody that specifically binds phosphorylated Thr88 of TDP-43; an antibody that specifically binds phosphorylated Ser91 of TDP-43; an antibody that specifically binds phosphorylated Ser92 of TDP-43; an antibody that specifically binds the combination of phosphorylated Thr88, phosphorylated Ser91 and phosphorylated Ser92 of TDP-43; an antibody that specifically binds phosphorylated Ser125 and phosphorylated Thr126 of TDP-43; an antibody that specifically binds phosphorylated Thr153 and phosphorylated Tyr155 of TDP-43; an antibody that specifically binds phosphorylated Thr153 of TDP-43; an antibody that specifically binds phosphorylated Tyr155 of TDP-43; an antibody that specifically binds phosphorylated Thr157 of TDP-43; an antibody that specifically binds phosphorylated Thr153 and phosphorylated Tyr155 of TDP-43; an antibody that specifically binds phosphorylated Tyr155 and phosphorylated Thr157 of TDP-43; an antibody that specifically binds phosphorylated Ser180 and phosphorylated Ser183 of TDP-43; an antibody that specifically binds phosphorylated Ser180 of TDP-43; an antibody that specifically binds phosphorylated Ser183 of TDP-43; and an antibody that specifically binds phosphorylated Ser292 of TDP-43. Epitope numbering of TDP-43 is in reference to accession number NP_031401.1 (see, SEQ ID NO:1).

Suitable methods for detecting a phosphorylated epitope of TDP-43 can be, for example, Western blot analysis, enzyme-linked immunosorbent assay (ELISA), immunoprecipitation, immunohistochemistry, and combinations thereof.

### EXAMPLES

### Example 1

In this Example, TDP-43 activity and structure change upon phosphorylation was analyzed.

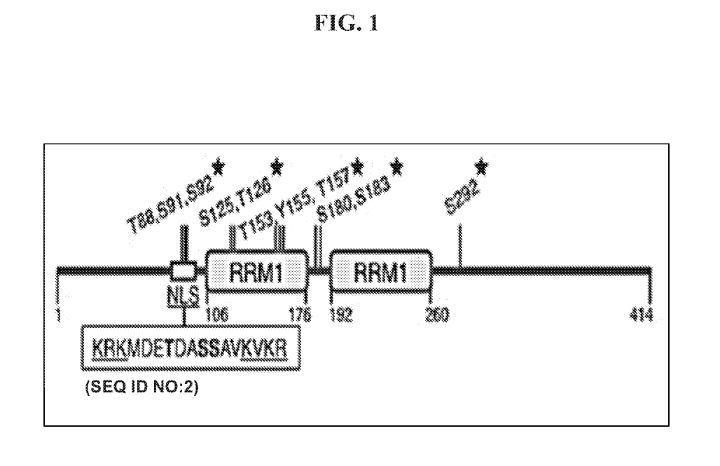

Modification of protein function/structure by phosphorylation is among the most common mechanisms of protein regulation in response to internal metabolic changes and to external environmental signals. These phosphorylation pathways are intensely investigated for their therapeutic potential in neurodegeneration and in other fields, such as cancer. The control of TDP-43 function and solubility by phosphorylation has not been well studied and thus, TDP-43 activity and structure change upon phosphorylation of specific amino acid residues was analyzed. Phosphorylation sites that have not been investigated were determined by mass spectrometry genome wide analyses following phosphorylated peptide enrichment under non-pathological conditions in human cells and mouse/rat tissue (Table 1). Specific residues at key regions of the protein that could potentially modify TDP-43 activity and structure were analyzed (FIG. 1). Table 2 describes the function associated with each of these regions.

TABLE 1
Domain Locations of TDP-43 Phosphorylation Sites numbering
is according to accession number NP_031401.1 (SEQ ID NO: 1))

| Phosphorylation Site | Domain Location | Domain Function |
|---|---|---|
| Thr88, Ser91, Ser92 | NLS (82-99) | Nuclear localization signal |
| | | essential to regulate RNA |
| | | processing |
| Ser125, Thr126, Thr153, | RRM1 (106-176) | Mediates RNA binding and |
| Tyr155, Thr157 | | is essential to regulate RNA |
| | | processing |
| Ser180, Ser183 | Linker (177-191) | Partly mediates RNA |
| | | binding specificity and |
| | | supports the structural |
| | | arrangement of the RRM |
| | | domains |
| Ser292 | Carboxy-terminal | Mediates protein-protein |
| | tail (261-414) | interactions that required to |
| | | regulate RNA processing |

Results demonstrated that phosphomimetic substitutions at RRM1 and in the linker region disrupted TDP-43 activity. This was at least partly due to decreasing RNA binding affinity in the presence of the phosphomimetic changes. Moreover, the decrease in RNA binding promoted TDP-43 aggregation in cells and in vitro. These results indicate that phosphorylation of RRM1 and linker region residues regulates TDP-43 interactions with its target RNA molecules and that increased levels of the phosphorylated forms may promote protein aggregation in cells. These features are commonly observed in TDP-43-associated disorders suggesting that aberrant control of the cellular phosphorylation pathways may occur during the disease process.

### Example 2

In this Example, polyclonal antibodies that specifically bind phosphorylated forms of the TDP-43 residues were developed.

Antibodies were designed that specifically bind to phosphorylated epitopes listed in Table 2. The antibodies are produced in rabbit following immunization with specific synthetic peptides.

Phosphospecific antibodies detecting phosphorylated serines 409/410 and 403/404 are widely used in the field and are commercially available (ProteinTech, Cosmo Bio, BioLegend). In contrast to the modifications in the studies presented in the present disclosure, phosphorylation of serines 409/410 and 403/404 is only detected in pathological samples and mostly used as markers for advanced stages of TDP-43 aggregation. The role of these modifications on the function and structure of TDP-43 and in the disease process has not been elucidated. In addition, antibodies made against phosphorylated serines 91, 92, 180, 183, 292, serines 91/92, and threonines 153, and 88 in addition to many other TDP-43 sites did not detect any forms of cellular or tissue-derived TDP-43 (Hasegawa, M., et al. (2008) Annals of Neurology, 64:60). Moreover, these antibodies were analyzed and were also found to be unable to detect TDP-43 protein.

TABLE 2
Antibodies to Phosphorylated TDP-43 (numbering is according
to accession number NP_031401.1 (SEQ ID NO: 1))
TDP-43 Phosphorylation Site

pThr88, pSer91, pSer92
pSer125, pThr126
pThr153, pTyr155
pThr153
pThr155
pThr157
pTyr155, pThr157
pSer180, pSer183
pSer180
pSer183
pSer292

What is claimed is:

1. An isolated antibody that specifically binds to a phosphorylated epitope of Transactive response DNA-binding protein 43 (TDP-43), wherein the phosphorylated epitope of TDP-43 is selected from the group consisting of Thr88, Ser91, Ser92, Ser125, Thr126, Thr153, Tyr155, Thr157, Ser180, Ser183, Ser292, and combinations thereof.

2. The antibody of claim 1, wherein the antibody is selected from the group consisting of a polyclonal antibody, a monoclonal antibody, a multispecific antibody, a human antibody, a humanized antibody, a camelised antibody, a chimeric antibody, single-chain Fv (scFv), single chain antibody, single domain antibody, a Fab fragment, a F(ab′) fragment, a disulfide-linked Fv (sdFv), and an anti-idiotypic (anti-Id) antibody.

3. The antibody of claim 1, wherein the antibody is conjugated to a detectable agent.

4. A method for detecting TDP-43 in a sample, the method comprising: contacting a sample with an antibody that specifically binds to a phosphorylated epitope of TDP-43, wherein the phosphorylated epitope of TDP-43 is selected from the group consisting of Thr88, Ser91, Ser92, Ser125, Thr126, Thr153, Tyr155, Thr157, Ser180, Ser183, Ser292, and combinations thereof, wherein the antibody and TDP-43 forms a complex; and detecting the complex.

5. The method of claim 4, wherein the complex is detected using a method selected from the group consisting of Western blot analysis, enzyme-linked immunosorbent assay (ELISA), immunoprecipitation, immunohistochemistry, and combinations thereof.

6. The method of claim 4, wherein the antibody is selected from the group consisting of a polyclonal antibody, a monoclonal antibody, a multispecific antibody, a human antibody, a humanized antibody, a camelised antibody, a chimeric antibody, single-chain Fv (scFv), single chain antibody, single domain antibody, a Fab fragment, a F(ab′) fragment, a disulfide-linked Fv (sdFv), and an anti-idiotypic (anti-Id) antibody.

7. A method for diagnosing a TDP-43-associated neurodegenerative disorder in a subject having or suspecting of having the TDP-43-associated neurodegenerative disorder, the method comprising: obtaining a sample from the subject; determining phosphorylation of TDP-43 in a sample by contacting the sample with an antibody that specifically binds a phosphorylated epitope of TDP-43, wherein the phosphorylated epitope of TDP-43 is selected from the group consisting of Thr88, Ser91, Ser92, Ser125, Thr126, Thr153, Tyr155, Thr157, Ser180, Ser183, Ser292, and combinations thereof; wherein the antibody and TDP-43 forms a complex; and detecting the complex.

8. The method of claim 7, wherein the TDP-43-associated neurodegenerative disorder is selected from the group consisting of amyotrophic lateral sclerosis, frontotemporal lobar degeneration, Alzheimer's disease, inclusion body myositis, Inclusion body myopathy associated with Paget's disease of the bone and frontotemporal dementia, limb-girdle muscular dystrophy, neuronal injury, and chronic traumatic encephalopathy.

9. The method of claim 7, wherein the complex is detected using a method selected from the group consisting of Western blot analysis, enzyme-linked immunosorbent assay (ELISA), immunoprecipitation, immunohistochemistry, and combinations thereof.

10. The method of claim 7, wherein the antibody is selected from the group consisting of a polyclonal antibody, a monoclonal antibody, a multispecific antibody, a human antibody, a humanized antibody, a camelised antibody, a chimeric antibody, single-chain Fv (scFv), single chain antibody, single domain antibody, a Fab fragment, a F(ab′) fragment, a disulfide-linked Fv (sdFv), and an anti-idiotypic (anti-Id) antibody.


SEQUENCE LISTING (2 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2 - protein, 17 aa, Homo sapiens
KRKMDETDASSAVKVKR